(19) United States
(12) Patent Application Publication
(10) Pub. No.: US 2018/0070853 A1
(43) Pub. Date: Mar. 15, 2018

(54) METHOD AND MAGNETIC RESONANCE APPARATUS FOR PLANNING A MAGNETIC RESONANCE MEASUREMENT OF A PATIENT EXAMINATION

(71) Applicant: Siemens Healthcare GmbH, Erlangen (DE)

(72) Inventors: Johannes Ehrl, Pinzberg (DE); Kirstin Jattke, Nuernberg (DE); Miriam Keil, Erlangen-Dechsendorf (DE); Christof Krellmann, Erlangen (DE); Stephan Nufer, Erlangen (DE); Peter Schmitt, Weisendorf (DE)

(73) Assignee: Siemens Healthcare GmbH, Erlangen (DE)

(21) Appl. No.: 15/699,516

(22) Filed: Sep. 8, 2017

(30) Foreign Application Priority Data
Sep. 13, 2016 (DE) 102016217421.5

(51) Int. Cl. A61B 5/055; G01R 33/30

(52) U.S. Cl. CPC A61B 5/0555; G01R 33/30

(57) ABSTRACT

In a method and MR apparatus for planning at least one MR measurement of an MR examination on a patient, the patient is positioned on a patient support within an examination room. A position of the patient support against movement of the patient support is secured within a patient receiving area of the MR scanner. Planning of the least one MR measurement of the MR examination for a first region of the patient to be examined takes place on the basis of the secured position of the patient support.

### BACKGROUND OF THE INVENTION

### Field of the Invention

The present invention concerns a method for planning a magnetic resonance (MR) examination of a patient using an MR apparatus, wherein the patient is positioned for this purpose on a patient support within an examination room. Furthermore, the invention concerns an MR apparatus for planning the MR examination of the patient, as well as a computer-readable data medium having code for implementing such a method.

### Description of the Prior Art

In magnetic resonance examinations it is usual for the region of a patient to be examined to be positioned in the isocenter, or in the immediate vicinity of the isocenter, of a magnetic resonance scanner. This makes it possible for the image data acquired to be of high quality. If a number of individual measurements are carried out consecutively on a patient, in which image data are to be acquired for different regions of the patient, the position of the patient support will be changed during this procedure so that, for example, the region of the patient to be examined is always located in an isocenter region of the magnetic resonance scanner for the upcoming magnetic resonance measurement. During planning of a magnetic resonance examination, however, the individual positions of the patient and/or of the patient support must be taken into account for the different magnetic resonance measurements in relation to the isocenter region.

By contrast, for magnetic resonance examinations during an intervention on the patient, for example, it is absolutely necessary for reasons of safety for the patient support apparatus always to maintain its position.

### SUMMARY OF THE INVENTION

An object of the present invention is to simplify planning of a magnetic resonance examination on a patient.

This object is achieved in accordance with the invention by a method for planning at least one magnetic resonance measurement of a magnetic resonance examination on a patient by operation of a magnetic resonance apparatus, wherein the patient is positioned for this purpose on a patient support within an examination room, the method having the following steps. A position of the patient support apparatus is secured against a movement of the patient support within a patient receiving area of the magnetic resonance scanner, and the at least one magnetic resonance measurement of the magnetic resonance examination is planned in a computer for a region of the patient to be examined with reference to the secured position of the patient support.

Preferably arranged within the examination room is the magnetic resonance scanner with a magnet unit, the patient receiving area, and the patient support. The magnet unit preferably has a magnet, a gradient coil and a radio-frequency antenna. Preferably the examination room is screened on the outside and inside in order to block disruptive influences, which can both influence a magnetic resonance examination and be caused by the magnetic resonance apparatus. For example, the examination room is screened both with respect to magnetic fields and against electromagnetic radiation, in particular radio-frequency radiation.

Preferably within the control room are all units and modules for control of the magnetic resonance apparatus. In particular the control room is screened from the examination room with respect to magnetic fields and against electromagnetic radiation, in particular radio-frequency radiation.

The patient support, in particular the patient table of the patient support, is preferably arranged so as to be moveable within the patient receiving area of the magnetic resonance scanner in the longitudinal direction of the patient table and/or of the patient receiving area. The securing of the position of the patient support against movement of the patient support is preferably done by an explicit user action. Preferably, the patient support is secured in a position in relation to the patient receiving area in which at least the first region of the patient to be examined is situated in the isocenter region of the magnetic resonance scanner.

The secured position of the patient support is preferably maintained during the entire magnetic resonance examination and cannot be changed, so that a higher safety standard can be provided during the magnetic resonance examination, in particular during an interventional magnetic resonance examination. Only after the secured setting has been released can the position of the patient support be changed in relation to the patient receiving area. In the secured position the patient support is locked against movement.

A simple and time-saving planning of a magnetic resonance examination is thereby possible, since the position of the patient support is already defined before the planning of the at least one magnetic resonance measurement of the magnetic resonance examination. Aborting a measurement of a magnetic resonance examination because of incorrect and/or imprecise planning, in which imprecise position specifications are present, can be prevented. This also enables the time that a patient spends within the magnetic resonance apparatus to be reduced to a minimum, and thus the RF load on the patient during a magnetic resonance examination, in particular an interventional magnetic resonance examination, to be lessened. A further advantage is that further magnetic resonance protocols from further magnetic resonance measurements can easily be added into the magnetic resonance examination as a function of the secured or locked position of the patient support.

In a further embodiment of the invention, the securing of the position of the patient support apparatus is initiated by a user action within a control room. This allows a simple and time-saving integration of the securing of the patient support apparatus into a planning workflow for planning of the magnetic resonance examination. The securing of the position of the patient support apparatus can be undertaken without the operating personnel having to leave the control room to do so, so that an interruption of the planning workflow is avoided.

Furthermore, before the step of securing the position of the patient support against movement of the patient support apparatus, position data of the patient can be acquired by a position determination measurement of the magnetic resonance apparatus, and the position of the first region of the patient to be examined is established with reference to the acquired position data.

The position data of the patient are preferably acquired by a position determination measurement or localizer measurement of the magnetic resonance apparatus. The position data accordingly are magnetic resonance data that allow the position of the patient to be determined therefrom, in particular the region of the patient to be examined. On the basis of the position data, the position and/or location of the patient within the patient receiving area is determined. The position of the first region to be examined can be determined on the basis of the position data in relation to the isocenter region of the magnetic resonance apparatus.

In an ideal position of the patient, the region of the patient to be examined is situated in the isocenter and/or an isocenter region of the magnetic resonance scanner. The isocenter is to be understood as meaning a point and/or a region that is located within the magnetic resonance scanner, in particular within the patient receiving area, in which the most ideal conditions exist within the magnetic resonance scanner for the magnetic resonance examination in the operation of the magnetic resonance apparatus. The isocenter is a point and/or a region at which the basic magnetic field of the magnetic resonance scanner is most homogeneous.

On the basis of the position data and/or the position of the first region of the patient to be examined, especially exact position information of the patient, in particular of the region to be examined, can be made available for the planning of the magnetic resonance examination. In addition, a user, in particular a member of the medical operating personnel, can check and/or test a position of the patient and/or of the first region of the patient to be examined, as to whether the current or acquired position matches a desired position and/or an examination position. This makes for a simple and efficient planning of the upcoming magnetic resonance examination possible, in particular of the at least one magnetic resonance measurement of the magnetic resonance examination. Advantageously, a correct examination position of the patient can already be available during the planning of the magnetic resonance examination.

In an embodiment of the invention, the position of the patient support to be secured against movement of the patient support causes the position of the first region to be examined to match an examination position. This achieves an especially efficient planning of the upcoming magnetic resonance examination, since a correct examination position of the patient already exists during the planning of the magnetic resonance examination. An undesired aborting of the measurement in a magnetic resonance examination because of an incorrect and/or imprecise position specification is avoided. This also enables the time that a patient spends within the magnetic resonance apparatus to be reduced to a minimum, and thus the RF load on the patient during an magnetic resonance examination, in particular an interventional magnetic resonance examination, to be lessened.

In the examination position, the patient support, in particular the patient table of the patient support, is situated within the patient receiving area such that the patient, in particular a region of the patient to be examined, is within the isocenter region.

Furthermore, before the position of the patient support is secured against movement of the patient support apparatus, a repositioning of the patient support apparatus can be done, if there is no match between the first region of the patient to be examined and the examination position of the patient. During the repositioning of the patient support, the patient support will be moved within the patient receiving area until there is a match between the first region of the patient to be examined and the examination position of the patient. The repositioning is preferably controlled automatically and/or autonomously by a control computer of the magnetic resonance apparatus, so that a high level of operating convenience can be achieved for the user of the magnetic resonance apparatus.

Because of the repositioning of the patient support, the patient support and thereby the patient, in particular the region of the patient to be examined, can be positioned ideally for the upcoming magnetic resonance examination before the planning of the upcoming magnetic resonance examination, so that a simple planning of the magnetic resonance examination for a user is achieved.

In another embodiment of the invention, the patient support has a locking assembly that locks the position of the patient support apparatus, wherein the securing of the position of the patient support is initiated by the locking assembly. Actuation of the locking assembly by a user enables the patient support to be locked and/or secured, or to be unlocked and/or released, from the secured position. This also makes possible a simple securing of the position of the patient support during, for example, preparation of the patient for the magnetic resonance examination. With interventional magnetic resonance examinations in particular, access for the intervention must also be provided, so that an ideal position, in which both the region to be examined is situated as close as possible to the isocenter region, and accessibility for interventional procedures is provided, is set within the examination room, in particular directly at the patient support.

After the securing of the position of the patient support by the locking assembly, position data of the patient can be acquired by a position determination measurement of the magnetic resonance apparatus, and the position of the first region of the patient to be examined is established on the basis of the position data. On the basis of the position data and/or the position of the first region of the patient to be examined, exact position information of the patient, in particular of the region to be examined, can be made available for the planning of the magnetic resonance examination. Simple and time-saving planning of the upcoming magnetic resonance examination, in particular of the at least one magnetic resonance measurement of the magnetic resonance examination, is thereby achieved.

In an embodiment of the invention, after the planning of the magnetic resonance examination, the at least one magnetic resonance measurement of the magnetic resonance examination is implemented in the secured position of the patient support apparatus. This enables an undesired premature aborting of the planned magnetic resonance examination, because undesired changes of position of the patient support apparatus are prevented. In addition, the examination time for the patient can be minimized, since delays as a result of time-consuming measurement aborts and restarts can be avoided.

Further in accordance with the invention, all magnetic resonance measurements of the magnetic resonance examination that were planned in the secured position of the patient support will be implemented with the patient support in the secured position. This enables a simple planning of complex magnetic resonance examinations with a number of individual magnetic resonance measurements to be achieved. A further advantage is that the magnetic resonance examination with the number of individual measurements can also be carried out safely and an undesired aborting of the magnetic resonance examination can be avoided.

In an embodiment of the invention, a second magnetic resonance measurement of the magnetic resonance examination is planned for a second region of the patient to be examined, the second region of the patient to be examined differing from the first region of the patient to be examined. The second magnetic resonance measurement is planned and carried out in the secured position of the patient support apparatus for the first region to be examined. In such cases, movement of the patient table during the magnetic resonance examination can be avoided and thus a high level of safety achieved during the magnetic resonance examination, in particular the interventional magnetic resonance examination. Additional magnetic resonance measurements can also be added during the planning phase or during the magnetic resonance examination, without boundary conditions for the magnetic resonance measurement, such as the position of the patient support, having to be acquired or entered in such cases, so that a flexible design of the magnetic resonance examination can be provided, in particular for interventional magnetic resonance examinations.

Especially advantageously, a secured table position can be maintained and thus a high level of safety guaranteed during the magnetic resonance examination, even if the second region of the patient to be examined is outside the isocenter region of the magnetic resonance apparatus.

Status information of the patient support apparatus can be displayed for a user at a display unit. The status information includes information as to whether the patient support apparatus is in a secured state or in an unsecured state. The status information can be represented, for example, by a lock symbol. Further presentation elements that appear reasonable to those skilled in the art are also possible can be included in the status information.

This means that the user, in particular a doctor, can always see whether the patient support is in a secured and/or locked state or in an unsecured and/or unlocked state, so that this status information can be taken into account by the user in the planning of the magnetic resonance examination. This also makes possible a simple planning of magnetic resonance examinations that includes both magnetic resonance measurements in which the patient support is secured in its position and magnetic resonance measurements in which the patient support is not secured in its position.

The secured position of the patient support can be displayed at a display unit, so that during the planning of the magnetic resonance examination by a user, for example a doctor, a simple and time-saving check of the position of the patient support, and thus of the patient position, can already be made.

In an embodiment of the invention, the secured position of the patient support in relation to an examination region of the patient is displayed by the display unit. This makes it possible, during the planning of the magnetic resonance examination, for the user already to perform a simple and time-saving check, in particular a visual check, of the position of the patient support apparatus in relation to the examination region of the patient.

The invention also encompasses a magnetic resonance apparatus, which has a patient support, a control computer, and a user interface with a display unit, the apparatus being designed to implement the method in accordance with the invention for planning at least one magnetic resonance measurement of a magnetic resonance examination on a patient.

This makes possible simple and time-saving planning of a magnetic resonance examination, since the position of the patient support is already defined before the planning of the at least one magnetic resonance measurement of the magnetic resonance examination. Aborting of a measurement of a magnetic resonance examination because of incorrect and/or imprecise planning, in which imprecise position specifications or incorrect position specifications are present, can advantageously be avoided. This also enables the time that a patient spends within the magnetic resonance apparatus to be reduced to a minimum and thus the RF load on the patient during a magnetic resonance examination, in particular of an interventional magnetic resonance examination, to be lessened. A further advantage is that further magnetic resonance protocols from further magnetic resonance measurements can easily be added into the magnetic resonance examination as a function of the secured and/or locked position of the patient support.

The advantages of the inventive magnetic resonance apparatus essentially correspond to the advantages of the inventive method for planning of at least one magnetic resonance measurement of a magnetic resonance examination on a patient, as described in detail above. Features, advantages and alternate embodiments mentioned above are applicable to the apparatus.

The patient support apparatus can have a locking assembly, so that securing the position of the patient support is initiated by the locking assembly, so a simple securing of the position of the patient support apparatus can be achieved, such as during a preparation of the patient for the magnetic resonance examination. The locking assembly of the patient support preferably operates only on the patient support. The locking assembly can be a mechanical locking assembly. Other embodiments of the locking assembly that appear reasonable to those skilled in the art are possible.

The present invention also encompasses a non-transitory, computer-readable data storage medium encoded with programming instructions (program code) that, when the storage medium is loaded into a control computer or computer system of a magnetic resonance apparatus, cause the control computer or computer system to operate the magnetic resonance apparatus in order to implement any or all of the above-described embodiments of the method according to the invention.

### BRIEF DESCRIPTION OF THE DRAWINGS

FIG. 1 shows an inventive magnetic resonance apparatus in a schematic illustration.

FIG. 2 shows a first exemplary embodiment of the inventive method for planning at least one magnetic resonance measurement of a magnetic resonance examination on a patient.

FIG. 3 shows a second exemplary embodiment of the inventive method for planning at least one magnetic resonance measurement of a magnetic resonance examination on a patient.

FIG. 4 shows a third exemplary embodiment of the inventive method for planning at least one magnetic resonance measurement of a magnetic resonance examination on a patient.

### DESCRIPTION OF THE PREFERRED EMBODIMENTS

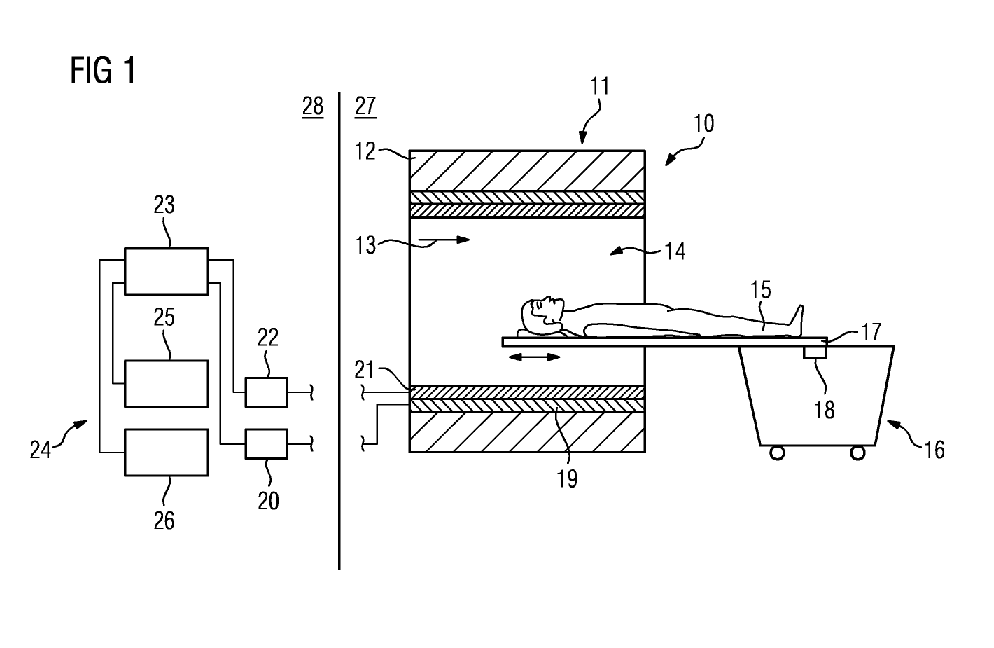

FIG. 1 is a schematic illustration of a magnetic resonance apparatus 10. The magnetic resonance apparatus 10 has a scanner 11, which has a superconducting basic field magnet 12 for creating a strong and constant basic magnetic field 13. The magnetic resonance scanner 11 has a patient receiving area 14 for receiving a patient 15. The patient receiving area 14 in the present exemplary embodiment is embodied in a cylindrical shape and is surrounded circumferentially by the scanner 11, in the shape of a cylinder. An embodiment of the patient receiving area 14 deviating therefrom is also conceivable.

The patient 15 can be moved into the patient receiving area 14 by a patient support 16. For this purpose, the patient support 16 has a patient table 17 designed so as to be able to be moved within the patient receiving area 14. The patient support 16 also has a locking assembly 18, with which a position of the patient table 17 can be locked, and thereafter unlocked. In a locked position of the patient table 17, the patient table 17 is secured in this position against movement of the patient table 17 in relation to the patient receiving area 14. Only in an unlocked state of the locking assembly 18 can the patient table 17 be moved within the patient receiving area 14.

The locking assembly 18 is arranged directly on the patient support 16. The locking assembly 18 preferably is a mechanical locking assembly 18, but other embodiments of the locking assembly 18 are possible.

The scanner 11 also has a gradient coil arrangement 19 that creates a magnetic field gradient, which is used for spatially encoding the MR signals during imaging. The gradient coil arrangement 19 is controlled by means of a gradient controller 20 of the magnetic resonance apparatus 10. The scanner 11 further has a radio-frequency antenna 21 is controlled by a radio-frequency antenna controller unit 22 so as to radiate a radio-frequency sequence into the patient receiving area 14. The radiated radio-frequency sequence gives certain nuclear spins in the patient 15 a magnetization that causes those nuclear spins to deviate from the field lines of the basic magnetic field 13, by an amount known as a flip angle. As these excited nuclear spins relax and return to the steady state, they emit the aforementioned MR signals. Those MR signals can be detected by the same RF antenna from which the radio-frequency sequence was radiated, or by a different RF antenna.

For controlling the basic field magnet 12, the gradient controller 20, and radio-frequency antenna controller 22, the magnetic resonance apparatus 10 has a control computer 23. The control computer 23 centrally controls the magnetic resonance apparatus 10, such as for executing a predetermined imaging gradient echo sequence. In addition the control computer 23 has an evaluation unit (not shown) for evaluation of medical image data that are acquired during the magnetic resonance examination.

Furthermore, the magnetic resonance apparatus 10 has a user interface 24, in communication with the control computer 23. Control information, such as imaging parameters, as well as reconstructed magnetic resonance images, can be displayed on a display unit 25, for example on at least one monitor, of the user interface 24 for medical operating personnel. Furthermore the user interface 24 has an input unit 26, via which information and/or parameters can be entered during a measurement process by the medical operating personnel.

The scanner 11 of the magnetic resonance apparatus 10 is situated together with the patient support 16 within an examination room 27. The control computer 23 and the user interface 24 are situated within a control room 28. The examination room 27 is screened on the inside and outside, in order to screen out disruptive influences, which can both influence a magnetic resonance examination and be caused by the magnetic resonance apparatus 10. For example, the examination room 27 is screened both with respect to magnetic fields and against electromagnetic radiation, in particular radio-frequency radiation. The control room 28 is also screened against magnetic fields and against electromagnetic radiation, in particular radio-frequency radiation, in relation to the examination room 27.

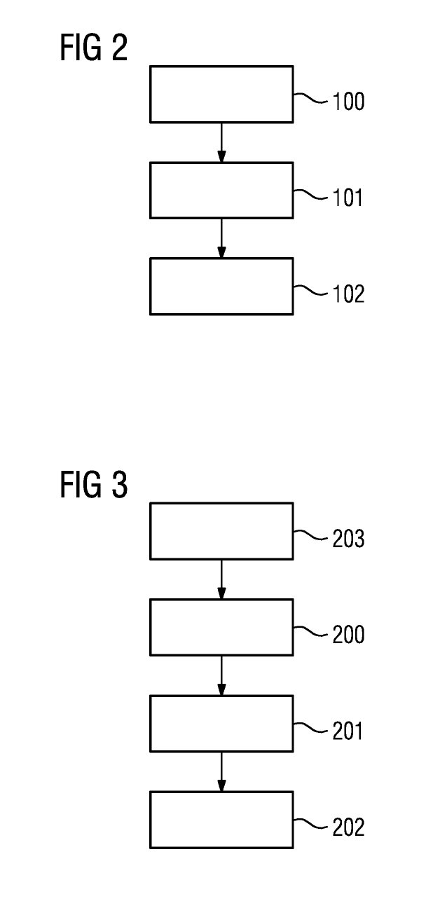

FIG. 2 shows a first exemplary embodiment of the inventive method for planning of at least one magnetic resonance measurement of a magnetic resonance examination on a patient 15. The method is controlled by the control computer 23, which accesses computer programs and/or software for this purpose, which are able to be loaded directly into a memory (not shown) of the control computer 23, with program code for implementing the method for planning at least one magnetic resonance measurement of a magnetic resonance examination on a patient, when the code is executed in the control computer 23. For this purpose, the control computer 23 has a processor designed to execute the computer programs and/or software. The computer programs and/or software also can be stored on an electronically readable data storage medium separately from the control computer 23. The data on the electronically readable data storage medium can be accessed by the control computer 23 when the storage medium is loaded into the computer 23.

In a first method step 100 of the method, a position of the patient support 16 is initially secured against movement of the patient support 16 within the patient receiving area 14 and/or in relation to the patient receiving area 14. When this is done, the patient 15 is already arranged on the patient support 16, in particular on the patient table 17 of the patient support 16, and is already prepared for the upcoming magnetic resonance examination.

The position of the patient support 16 is preferably secured against movement of the patient support 16 by an explicit user action. The user action can be actuated from the control room 28, via the user interface 24, in particular the input unit 26 of the user interface 24. The position of the patient support apparatus 16 can be secured against a movement of the patient support apparatus 16 from the examination room 27 by actuation of the locking assembly 18.

In the secured locked state of the patient support 16, the patient support 16 can no longer be moved, so that it has a fixed position within the patient receiving area 14. In an unsecured and/or unlocked state of the patient support 16, the patient support 16 can be brought into any given position within the patient receiving area 14.

In a subsequent method step 101, planning of the at least one magnetic resonance measurement of the magnetic resonance examination takes place for a first region of the patient to be examined 15, on the basis of the secured position of the patient support apparatus 14. The planning step is undertaken by the control computer 23, and the user can enter parameter values, and/or define a sequence for a number of magnetic resonance measurements of the magnetic resonance examination etc., via the user interface 24, in particular via the input unit 26 and the display unit 25.

To do this, status information of the patient support apparatus 16 is first displayed for the user at the display unit 25. The status information includes information as to whether the patient support 16 is in the secured and/or locked state or whether the patient support 16 is in the unsecured and/or unlocked state. The status information can be displayed to the user, in particular to the medical operating personnel as a lock symbol and/or further display elements at the display unit 25. A closed lock symbol, for example, can indicate a secured and/or locked state of the patient support 16. An open lock symbol then indicates an unsecured and/or unlocked state of the patient support 16. In this way the user will be shown current status information at all times during the planning step.

As well as the status information of the patient support 16, the user can also be shown the secured position of the patient support apparatus 14 at the display unit 25. The secured position of the patient support apparatus 16 is shown in relation to an examination region of the patient 15 at the display unit 25.

During the method step 101, two or more magnetic resonance measurements of the upcoming magnetic resonance examination can be planned. Here a second magnetic resonance examination for a second region of the patient 15 to be examined can be planned, wherein the second region of the patient 15 to be examined is different from the first region of the patient to be examined 15. If the planning of the second magnetic resonance measurement is done on the basis of the position data of the secured position of the patient support 16, the second magnetic resonance measurement is also based on the position data of the secured position of the patient support 16. In this case, the second region of the patient 15 to be examined can also be outside an isocenter region of the magnetic resonance scanner 11.

Subsequently, in a further method step 102, the planned magnetic resonance measurement of the magnetic resonance examination is implemented. Here all magnetic resonance measurements of the magnetic resonance examination for which the position data of the secured patient support 16 was the basis in the planning, are also carried out in the secured position of the patient support 16.

FIG. 3 shows an alternate exemplary embodiment of the method for planning of at least one magnetic resonance measurement of a magnetic resonance examination on a patient. Components, features and functions that essentially remain the same are basically labeled with the same reference characters. The description given below is essentially restricted to the differences from the exemplary embodiment in FIGS. 1 and 2, wherein, as regards components, features and functions that remain the same, the reader is referred to the description of the exemplary embodiment in FIGS. 1 and 2.

The second exemplary embodiment of the method is controlled by the control computer 23, which has computer programs and/or software for this purpose, which are able to be loaded directly into a memory of the control computer 23 with program code for carrying out the inventive method, when the program code is executed in the control computer 23. For this purpose the control computer 23 has a processor which is designed to execute the computer programs and/or software. As an alternative, the computer programs and/or software can be stored on an electronically readable data storage medium embodied separately from the control computer 23. The data on the electronically readable data storage medium can be accessed by the control computer 23 when the storage medium is loaded into the computer 23.

In a first method step 203 of the method, there is initially an acquisition of position data of the patient 15 by execution of a position data measurement of the magnetic resonance apparatus 10. With the position data measurement and/or a localizer measurement, magnetic resonance data for determining the position of the patient 15 are acquired. On the basis of the acquired position data, a position of a first region of the patient to be examined 15 will subsequently be established automatically by the control computer 23.

If the position of the first region of the patient to be examined 15 matches an examination position, subsequently, in a further method step 200, the patient support 16 is secured against movement of the patient support 16.

If there is no match between the position of the region of the patient to be examined 15 and the examination position of the patient 15, before the method step 200 of securing the position of the patient support 16 against movement of the patient support 16, a repositioning of the patient support 16 is carried out. The repositioning of the patient support 16 is still done in the method step 203 of acquiring position data of the patient 15. The repositioning of the patient support 16 is done on the basis of the acquired position data. In addition, the repositioning of the patient support 16 by the control computer 23 is carried out automatically and/or autonomously, so that the medical operating personnel do not have to leave the control room 28 to do so.

Subsequently, in the method step 200, the position of the patient support 16 is secured against movement of the patient support 16 in relation to the patient receiving area 14. Here the position of the patient support 16 is preferably secured by an explicit user action, which is initiated within the control room 28 via the user interface 24, in particular the input unit 26 of the user interface 24.

In a following method step 201, planning of the at least one magnetic resonance measurement of the magnetic resonance examination for the first region of the patient to be examined 15 takes place on the basis of the secured position of the patient support 16. The planning step is undertaken by the control computer 23, wherein the user can enter parameter values and/or define a sequence, for a number of magnetic resonance measurements of the magnetic resonance examination etc., via the user interface 24, in particular via the input unit 26 and the display unit 25.

To do this, status information of the patient support apparatus 16 is first displayed for the user at the display unit 25. The display of the status information, as well as a scope of the status information, correspond in this case to the information given about the display of the status information relating to FIG. 2 (the first exemplary embodiment).

As well as the status information of the patient support 16, the user can be shown the secured position of the patient support 16 at the display unit 26. The secured position of the patient support 16 in relation to an examination region of the patient 16 is shown at the display unit 26.

During the method step 201, two or more magnetic resonance measurements of the upcoming magnetic resonance examination can also be planned. Here a second magnetic resonance examination can be planned for a second region of the patient 15 to be examined, wherein the second region of the patient 15 to be examined is different from the first region of the patient to be examined 15. If the second magnetic resonance measurement is planned here on the basis of the position data of the secured position of the patient support 16, the second magnetic resonance measurement is also based on the position data of the secured position of the patient support 16. In this case the second region of the patient 15 to be examined can also be arranged outside the isocenter region of the magnetic resonance 10.

Thereafter, in a further method step 202, the planned magnetic resonance measurement of the magnetic resonance examination is carried out. Here all magnetic resonance measurements of the magnetic resonance examination, for which in the planning of the position data of the secured patient support 16 was the basis, are also carried out in the secured position of the patient support 16.

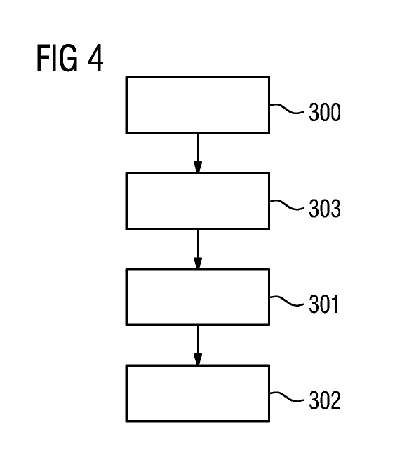

FIG. 4 shows an alternate exemplary embodiment of the inventive method. Components, features and functions that essentially remain the same are basically labeled with the same reference characters. The description given below is essentially restricted to the differences from the exemplary embodiment in FIGS. 1 and 2, wherein, as regards components, features and functions that remain the same, the reader is referred to the description of the exemplary embodiment in FIGS. 1 and 2.

The third exemplary embodiment of the method for planning of at least one magnetic resonance measurement of a magnetic resonance examination on a patient 15 is controlled by the control computer 23, which has computer programs and/or software for this purpose, which are able to be loaded directly into a memory of the control unit 23 with program code for carrying out the method when the computer programs and/or software are executed in the control computer 23. For this purpose the control computer 23 has a processor designed to execute the computer programs and/or software. As an alternative, the computer programs and/or software can be stored on an electronically readable data storage medium embodied separately from the control computer 23, wherein the data on the electronically readable data storage medium can be accessed by the control computer 23 when the storage medium is loaded into the computer 23.

In a first method step 300 of the method, a securing of a position of the patient support 16 against movement of the patient support 16 within the patient receiving area 14 and/or in relation to the patient receiving area 14 takes place. The securing of the position of the patient support 14 against movement of the patient support 14 is preferably undertaken by an explicit user action. The user action can be actuated from the examination room 27 by the locking assembly 18.

Thereafter, in a further method step 303, position data of the patient 15 are acquired by a position determination measurement and/or localizer measurement of the magnetic resonance apparatus 10, and a position of a first region of the patient to be examined 15 is established on the basis of the acquired position data. Subsequently, in a further method step 301, the at least one magnetic resonance measurement of the magnetic resonance examination is planned for the first region of the patient to be examined 15 on the basis of the secured position of the patient support apparatus 16. The planning step is undertaken by the control computer 23, wherein the user can enter parameter values and/or define a sequence for a number of magnetic resonance measurements of the magnetic resonance examination etc. via the user interface 24, in particular via the input unit 26 and the display unit 25.

To do this, status information of the patient support apparatus 16 is first displayed for the user at the display unit 25. The display of the status information as well as a scope of the status information correspond in this case to the information given about the presentation of the status information relating to FIG. 2 (the first exemplary embodiment).

As well as the status information of the patient support 16, the user can also be shown the secured position of the patient support 16 at the display unit 25. The secured position of the patient support 16 is shown in relation to the examination region of the patient 15 at the display unit 25.

During the method step 301, two or more magnetic resonance measurements of the upcoming magnetic resonance examination can also be planned. Here a second magnetic resonance examination can be planned for a for a second region of the patient 15 to be examined, wherein the second region of the patient 15 to be examined 15 is different from the first region of the patient to be examined 15. If the second magnetic resonance measurement is planned here on the basis of the position data of the secured position of the patient support 16, the second magnetic resonance measurement is also based on the position data of the secured position of the patient support 16. In this case the second region of the patient 15 to be examined can also be outside an isocenter region of the magnetic resonance apparatus 10.

Thereafter, in a further method step 302, the planned magnetic resonance measurements of the magnetic resonance examination are carried out. Here all magnetic resonance measurements of the magnetic resonance examination, for which the position data of the secured patient support 16 was the basis in the planning, are also carried out in the secured position of the patient support 16.

In an alternate embodiment of the magnetic resonance apparatus 10 and/or of the method for planning of at least one magnetic resonance measurement of a magnetic resonance examination on a patient 15, the user interface 24 can also be embodied separately from the magnetic resonance apparatus 10. Here the user interface 25 can be provided by a mobile terminal, for example.

Although modifications and changes may be suggested by those skilled in the art, it is the intention of the Applicant to embody within the patent warranted hereon all changes and modifications as reasonably and properly come within the scope of the Applicant's contribution to the art.

1. A method for planning at least one magnetic resonance (MR) measurement of an MR examination of a subject using an MR apparatus having an MR data acquisition scanner, wherein the patient is positioned for said measurement on a patient support within an examination room in which the MR scanner is situated, said method comprising:
securing a position of the patient support against a movement of the patient support within a patient-receiving area of the MR scanner; and
in a computer, planning said at least one MR measurement of the MR examination for a region of the subject to be examined, based on the secured position of the patient support, thereby producing a planned MR examination, and making a representation of the planned MR examination available from the computer in electronic form as an output of said computer.

2. A method as claimed in claim 1 comprising securing the position of the patient support by initiating a user action from within a control room that is physically separated from said examination room.

3. A method as claimed in claim 1 comprising, before securing the position of the patient support against movement of the patient support, operating said MR apparatus to execute a position determine measurement in which patient data are acquired from the patient and, in said computer, determining a position, from said position data, of the region of the patient to be examined, and using said position of said region to produce said planned MR examination.

4. A method as claimed in claim 3 comprising securing the patient support against movement of the patient support by fixing said patient support at a position relative to said examination area that causes said region of the patient to be examined to match a predetermined examination position.

5. A method as claimed in claim 3 comprising, before securing the patient support against movement of the patient support, determining, in said computer, whether the position of the region of the patient to be examined matches a predetermined examination position and, if not, re-positioning the patient support.

6. A method as claimed in claim 1 wherein said patient support comprises a locking assembly operable to lock the patient support in a position, and wherein securing the position of the patient support comprises actuating said locking assembly.

7. A method as claimed in claim 6 comprising, after securing the position of the patient support by actuation of said locking assembly, operating the MR data acquisition scanner to execute a position determination measurement in which patient data of the patient are acquired and, in said computer, deriving, from said position data, a position of the region of the patient to be examined.

8. A method as claimed in claim 1 comprising operating said MR data acquisition scanner to execute said planned MR examination, with the patient support in the secured position.

9. A method as claimed in claim 1 wherein said MR examination comprises a plurality of MR measurements, and including all of said MR measurements in said planned MR examination, and operating said MR data acquisition scanner to execute all of said MR measurements with said patient support in said secured position.

10. A method as claimed in claim 1 wherein said region of the patient is a first region of the patient and said MR measurement is a first MR measurement of said first region of the patient, and comprising generating said planned MR examination to include a second MR measurement of a second region of the patient, which differs from said first region, and operating said MR data acquisition scanner to execute said planned MR examination with said patient support in said secured position for each of said first and second MR measurements.

11. A method as claimed in claim 10 comprising placing said support apparatus in an examination position, as said secured position, that situates said first region of the patient in an isocenter region of the MR data acquisition scanner, and that situates said second region of the patient outside of said isocenter region.

12. A method as claimed in claim 1 comprising, from said computer, displaying status information about the patient support at a display in communication with said computer, said status information identifying whether the patient support is in a secured state or in an unsecured state.

13. A method as claimed in claim 1 comprising, at a display in communication with said computer, displaying said secured position of the patient support relative to said MR data acquisition scanner.

14. A method as claimed in claim 13 comprising displaying said secured position of the patient support in relation to the region of the patient to be examined.

15. A magnetic resonance (MR) apparatus comprising:
an MR data acquisition scanner;
a patient support adapted to receive a patient thereon, said patient support, or a portion thereof, being moveable relative to said MR data acquisition scanner;
a computer configured to generate an electronic command that secures a position of the patient support against a movement of the patient support within a patient-receiving area of the MR scanner; and
said computer being configured to plan said at least one MR measurement of the MR examination for a region of the subject to be examined, based on the secured position of the patient support, thereby producing a planned MR examination, and to make a representation of the planned MR examination available from the computer in electronic form as an output of said computer.

16. An MR apparatus as claimed in claim 15 wherein said patient support comprises an actuatable locking assembly, and wherein said patient support is configured to be placed in said secured position by actuation of said locking assembly.

17. A non-transitory, computer-readable data storage medium encoded with programming instructions, said storage medium being loaded into a computer system of a magnetic resonance (MR) apparatus that comprises an MR data acquisition scanner and a patient support, adapted to receive a patient thereon, that is moveable relative to said MR data acquisition scanner, said programming instructions causing said control computer to:
emit a command that secures a position of the patient support against a movement of the patient support within a patient-receiving area of the MR scanner; and
plan said at least one MR measurement of the MR examination for a region of the subject to be examined, based on the secured position of the patient support, thereby producing a planned MR examination, and make a representation of the planned MR examination available from the computer in electronic form as an output of said computer.
